(19) United States
(12) Patent Application Publication
(10) Pub. No.: US 2015/0335919 A1
(43) Pub. Date: Nov. 26, 2015

(54) PHASED ARRAY ENERGY AIMING AND TRACKING FOR ABLATION TREATMENT

(71) Applicant: PERSEUS-BIOMED INC., Orangeburg, NY (US)

(72) Inventors: Boaz BEHAR, Ganei-Tikva (IL); Yoni HERTZBERG, Moshav Ben-Shemen (IL)

(21) Appl. No.: 14/758,640

(22) PCT Filed: Dec. 31, 2013
(86) PCT No.: PCT/IB2013/061457

§ 371 Date: Jun. 30, 2015

Related U.S. Application Data
(60) Provisional application No. 61/747,465, filed on Dec. 31, 2012.

(51) Int. Cl. A61N 7/02; A61B 19/00; A61B 6/00; A61B 6/04; A61B 6/12

(52) U.S. Cl. CPC A61N 7/02; A61B 6/0492; A61B 6/12; A61B 6/487; A61B 19/5225; A61B 2019/5238; A61B 2019/5437; A61B 2019/5466

(57) ABSTRACT

A method for transmitting an energy beam from a group of elements of an external phased array energy projector for a medical ablation treatment. The method comprises transmitting first energy pulses from a subset of elements of the group, measuring signal parameters of first energy signals from reception of first energy pulses by energy sensors, and calculating for the group energy transmission parameters. The method further comprises transmitting second energy pulses by other elements, measuring signal parameters of second energy signals based on a reception the pulses by energy sensors, and calculating for the group a second set of energy transmission parameters. The method further comprises calculating transmission instructions for transmitting a phased array energy beam from the group by combining first and second energy transmission parameters. The method transmits an energy beam based on the transmission instructions.

### RELATED APPLICATIONS

This application also incorporates by reference the disclosures of International PCT Patent Applications Nos. PCT/IB2012/054524 and/or PCT/IB2012/054525, both filed on Sep. 2, 2012.

### FIELD AND BACKGROUND OF THE INVENTION

The present invention, in some embodiments thereof, relates to medical tissue ablation procedures and, more particularly, but not exclusively, to energy aiming and tracking for renal sympathetic denervation.

In renal denervation (RDN) pharmacological-resistant hypertension may be treated by ablating the nerves to the renal arteries to lower chronically high blood pressure. An estimated 30% of patients with hypertension are resistant to pharmacological treatment. As an alternative to pharmacological treatment, RDN has been used to lower average blood pressure by approximately 30 mm Hg, measured three years following treatment. Systems to perform RDN use either catheters positioned in the renal arteries to deliver energy to the surrounding nerves, or extracorporeal radiated energy from transmitters, emitters and/or transducers. The radiated energy heats the tissue surround the arteries and thus ablates the sympathetic nerves leading to these arteries. The physiological response to ablating these nerves may be a lowering of the blood pressure. The extracorporeal RDN systems are based on radiofrequency electromagnetic (RFEM) or ultrasonic (US) energy delivery devices. The US energy delivery devices are also called high intensity focused ultrasound (HIFU) devices, and may incorporate an imaging modality, such as magnetic resonance imaging or ultrasound imaging, to assist in the procedure.

Existing methods for transmitting a beam of energy from a phased array energy projector during a tissue ablation procedure rely on non-invasive detection and imaging methods to determine when the energy reaches the target location and for tracking any target location motion. Imaging modalities and/or sensors may be used to measure the heat produced by energy beams and experimentally attempt to better focus the energy beam on the treatment area. For example, magnetic resonance imaging are used with tissue segmentation to estimate the transmission parameters of each element of the phased array transducer to best focus the beam of energy, as well as measure the temperature of the target tissue during treatment.

For example, U.S. Pat. No. 8,372,009 describes a targeting catheter used to locate an arteriotomy, such as is performed during a femoral artery catheterization procedure. The targeting catheter uses a transmitter or receiver to perform time of flight measurements to locate a therapeutic ultrasonic applicator relative to the target.

Another example is described in U.S. Pat. No. 8,295,912 that describes a method and system to inhibit a function of a nerve traveling with an artery, optionally using a catheter based heat and/or temperature sensor.

### SUMMARY OF THE INVENTION

According to some embodiments of the present invention there is provided a method for transmitting a phased array energy beam from an energy transmission group of elements of a phased array energy projector external to a body of a target patient, comprising the following actions. The method comprises transmitting one or more first energy pulse from one or more phased array element from two or more phased array elements of a phased array energy projector external to a target patient, measuring one or more signal parameter of one or more first energy signal based on a reception of the one or more first energy pulse by one or more energy sensor, and calculating for each member of an energy transmission group of the plurality of phased array elements a first energy transmission parameter based on the one or more signal parameter. The method further comprises transmitting one or more second energy pulse by one or more of the plurality of phased array elements, measuring one or more signal parameter of one or more second energy signal based on a reception of the one or more second energy pulse by one or more energy sensor, and calculating for each member of the energy transmission group a second energy transmission parameter according to the one or more signal parameter. The method further comprises calculating transmission instructions for transmitting a phased array energy beam toward an intrabody target area in a body of the target patient from the energy transmission group by combining the first and second energy transmission parameters for each member of the energy transmission group. The method further comprises controlling the phased array energy projector to transmit the phased array energy beam from the energy transmission group based on the transmission instructions.

Optionally, the first and second energy transmission parameters for each phased array element of the energy transmission group are any from the list of phased values, amplitude values, frequency values, time values, and the like.

Optionally, the transmission instructions for each phased array element of the energy transmission group are any from the list of phased values, amplitude values, frequency values, time values, and the like.

Optionally, the one or more energy sensor is placed in proximity to the intrabody treatment area of the target patient using any from the list of one or more catheter, one or more endoscopy, one or more wireless capsule endoscope, one or more hypodermic needle, one or more biopsy needle, one or more biopsy probe, and the like.

Optionally, the signal parameter of the one or more first energy signal is a measured phase value of the one or more first energy signal, where the plurality of phased array elements used to transmit the one or more first energy pulse is a subset of the energy transmission group, and the calculating is performed by extrapolation and interpolation of the phase values of the subset.

Optionally, the signal parameter of the one or more second energy signal is a measured phase value of the one or more second energy signal, and the one or more second energy signal is collected for all remaining elements of the energy transmission group.

Optionally, some of the signal parameters of the one or more first energy signal is a time of flight value of the one or more second energy signal, and the time of flight value is used tissue modeling to calculate a phase value.

Optionally, the one or more phased array energy projector is any type from the list of high intensity ultrasound phased array, radiofrequency electromagnetic phased array, gamma radiation phased array, proton therapy phased array, and the like, and the one or more energy sensor is configured to detect the projected energy of the phased array energy projector.

Optionally, the transmission instructions are calculated for transmitting the phased array energy beam toward an intrabody moving target area by combining previously determined transmission instructions with a repeated transmitting of the one or more first energy pulse, measuring the one or more signal parameter of the one or more first energy signal for a subset of elements of the energy transmission group.

According to some embodiments of the present invention there is provided a method for transmitting a phased array energy beam from a phased array energy projector external to a body of a target patient comprising the following actions. The method comprises projecting one or more energy pulse from one or more element of a phased array energy projector located external to a body of a target patient, receiving two or more sensor signals from each of the one or more energy pulses using two or more energy sensors located in a body of the target patient in proximity to an intrabody treatment area of the target patient, and measuring a phase value from each of the plurality of sensor signals. The method further comprises calculating for each member of an aiming group of the one or more phased array elements one or more energy transmission parameter adjustment based on the phase values, one or more known distance between the energy sensors, and one or more relative position of one or more target treatment location. The method further comprises calculating transmission instructions for transmitting a phased array energy beam based on previous transmission instructions and the one or more energy transmission parameter adjustment, and controlling the phased array energy projector to transmit the phased array energy beam from the aiming group based on the transmission instructions.

Optionally, the one or more relative position is two or more relative positions defining one or more ablation point-by-point pattern.

Optionally, the one or more relative position is two or more continuous relative positions located along one or more ablation path defining one or more ablation sweep pattern.

According to some embodiments of the present invention there is provided a computerized device for automatically guiding an energy beam from a phased array energy projector comprising the following components. The device comprises, one or more user interface, one or more interface for one or more energy sensor, one or more interface for one or more phased array energy projector. The device further comprises one or more processing unit, configured for projecting energy from one or more element of the one or more phased array energy projector, receiving one or more sensor signal from the one or more energy sensor, computing one or more signal value from the one or more sensor signal, and guiding one or more energy beam from the phased array energy projector automatically, using the one or more signal value.

Optionally, the one or more signal value is a time of flight value.

Optionally, the one or more signal value is a phase value.

Optionally, the guiding comprises automatically focusing the one or more energy beam.

Optionally, the guiding comprises automatically aiming the one or more energy beam to one or more target treatment location in point-by-point pattern of ablation locations.

Optionally, the guiding comprises automatically aiming the one or more energy beam to one or more target treatment location in sweep pattern of ablation locations.

Optionally, the guiding comprises automatically tracking one or more new target treatment location and automatically aiming the one or more energy beam to the one or more new target treatment location.

Optionally, the one or more user interface comprises a three dimensional image of a ablation treatment plan in a coordinate system relative to the target treatment anatomy and the one or more energy sensor, enabling a user to instruct the device to perform modifications to the ablation treatment plan.

According to some embodiments of the present invention there is provided a medical treatment method for performing an ablation treatment comprising the following actions. The medical method comprises preparing a patient in a treatment suite, inserting one or more energy sensor into one or more treatment region of the patient, and positioning one or more phased array energy projector so that projected energy beam reaches the one or more treatment region. The medical method further comprises initiating an automatic computerized system for focusing and aiming a projected energy beam, the focusing and aiming performed using a projection of one or more low power energy pulse from one or more element of the one or more phased array energy projector, receiving one or more intrabody sensor signal from the one or more low power energy pulse, and computing one or more energy transmission value from the one or more intrabody sensor signal, and performing an ablation treatment.

Optionally, the ablation treatment incorporates automatic tracking of a location of the one or more treatment region, the automatic tracking performed using a projection of one or more low power energy pulse from one or more element of the one or more phased array energy projector, receiving one or more intrabody sensor signal from the one or more low power energy pulse, and computing one or more energy transmission value from the one or more intrabody sensor signal.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of a method for renal denervation, according to some embodiments of the invention;

FIG. 2 is a flowchart of a method for focusing and aiming a phased array energy projector, according to some embodiments of the invention;

FIG. 3 is a flowchart of a method for tracking a treatment location to aim a phased array energy projector, according to some embodiments of the invention;

FIG. 4 is a schematic illustration of a computerized device for renal denervation, according to some embodiments of the invention;

FIG. 5 is a schematic illustration of a graphical user interface for renal denervation, according to some embodiments of the invention;

FIG. 6 is a schematic illustration of a system for renal denervation, according to some embodiments of the invention;

FIG. 7 is a schematic illustration of a system for renal denervation in a catheterization laboratory, according to some embodiments of the invention;

FIG. 8 is a schematic illustration of a blood vessel and catheter attached sensors for renal denervation, according to some embodiments of the invention;

FIG. 9 is a schematic illustration of ablation point patterns for renal denervation, according to some embodiments of the invention; and

FIG. 10 is a schematic illustration of ablation sweep patterns for renal denervation, according to some embodiments of the invention.

FIG. 11 is a schematic illustration of a phased array energy projector, according to some embodiments of the invention.

### DESCRIPTION OF EMBODIMENTS OF THE INVENTION

The acoustic properties of different tissues may differ substantially, and may differ in the range of 10% in the soft tissues of the same person. This behavior may cause ultrasound waves to distort when passing through areas of inhomogeneous tissue in accordance with Snell's law of refraction. As a result, ultrasound energy transmission from any type of transducer to any target which is far enough from the transducer may result in the beam focus deviating from its intended location, for the focal spot to defocus and/or blur, become larger, and have reduced energy density.

When tracking the target treatment location using external, non-invasive methods, the resulting tissue speed of sound modulations will prevent accurate positioning of the focal spot at the target treatment location, compounded with the previously described focal spot aberrations.

An energy beam from a phased array transducer may be focused by directly measuring the phase of a received signal at an energy sensor near the target treatment location, as described in PCT/IB2012/054525. But the phased array may contain thousands of elements and to directly measure the phase value of a signal received at an energy sensor for each element may require hundreds of seconds. During this focusing time, target treatment location motion may cause the directly measured phase values at the energy sensors to be based on different locations and resulting in blurring of the focal spot. Motion tracking during the focusing measurements for the energy beam may be used to focus the energy beam during treatment.

By locating at least one energy sensor inside the patient in close proximity to the target treatment location, the tissue speed of sound modulations of each phased array element may be corrected. An energy pulse may be sent from each element of the extracorporeal phased array, the signal received from the energy pulse may be detect by the energy sensors at the treatment location, and a phase value the signal may be measured directly. The tissue speed of sound modulations for the treatment location of that specific patient may be corrected by adjusting the transmission phase value of each phased array element based on the corresponding directly measured phase value of the sensor signal. The adjusted transmission phase value of the energy projection from each phased array element during treatment may produce a more accurate focus and aiming of the energy beam from the phased array transducer than non-invasive imaging and detection methods.

Receiving projected energy from elements of a phased array ultrasonic transducer at one or more energy sensors located near the target treatment location may be used to automatically adjust the energy transmission phase values of each phased array transducer element to aim the focal spot of the energy beam to a relative location near the energy sensors. These transmission energy phase value adjustments may position the energy beam focal spot relative to the energy sensors without computation of distances between the phased array transducer and the treatment location, without knowledge of the actual location of the one or more sensors, and without need for detailed knowledge of the tissue anatomy of that patient. This focal spot aim adjustment may allow planning an effective ablation treatment pattern and avoiding damage to healthy tissue surrounding the treatment target location.

Using signals received at the energy sensors during the energy projection from the phased array transducer may automatically correct for the target treatment location motion during the focusing of the energy beam and also automatically track physiological and target treatment location motion during the treatment.

A multiple stage method may track target motion during focusing and treatment by performing transmission phase value corrections at each stage of increased accuracy and decreased scale. One stage may include time of flight measurements of projected energy pulses between some small number of the phased array energy projector and the energy sensors. One stage may include direct measurement of phase correction on a representative set of the phased array elements and interpolate and/or extrapolate the transmission phase values from the measured elements to all elements of the phased array transducer.

The increased accuracy of the size, boundaries and positioning of the beam focus may enable decreased treatment time, resurgery and adverse effects as well as increased positive outcome from treatment and patient comfort.

According to some embodiments of the present invention, there are provided methods and devices to determine focusing of an energy beam in multiple stages. During each stage, energy is projected from one or more elements of a phased array energy projector, a signal from the projected energy is received at the one or more energy sensors near the treatment location, a signal value is calculated from the sensor signal to measure the time of flight, phase, and/or any other property of the received signal, and the signal values from multiple phased array elements are used to compute the transmission phase values of all elements of the phased array transducer. Using TOF values with or without tissue modeling may correct for target motion. Using direct measurement of phase values may also correct for target motion. The energy projection from one of more elements of the phased array may be done serially, and during each successive stage other elements used to further refine the computed transmission phase values.

For example, during a first stage a time of flight (TOF) value may be computed from the sensor signal for less than 0.5% of the transducer elements and converted to an expected phase value for all elements of the phased array energy projector using tissue modeling. In this example, during a second stage a phase value may be computed from the sensor signal for less than 5% of the phased array elements, the phase value converted to a transmission phase value for the transducer elements, and the transmission phase value extrapolated and/or interpolated to all elements of the phased array. In this example, during a third stage a phase value may be computed from the sensor signal for all of the phased array elements in batches, each batch containing between 5 and 10% of the total number of elements in the phased array transducer. Prior to each batch of the third stage, the first and second stages may be repeated to track motion of the energy sensor during the focusing of the energy beam. The energy sensors may be in a fixed position relative to the target treatment location so motion of the energy sensors may be equivalent to the motion of the target treatment location. Combining the transmission phase values of the three stages for each element of the phased array transducer may focus the energy beam. The number of phased array elements used for each stage may be determined by the ultrasound transmission frequency, the time each measurement takes to travel in the tissue and be distinct from a following measurement, and computation time for each stage, and the desired temporal resolution of the motion tracking.

According to some embodiments of the present invention, there are provided methods and devices for automatically utilizing energy sensors in proximity to a treatment location to determine automatically the phased array transducer element transmission phase value adjustment for targeting a point in the region surrounding one or more cavity and/or lumen inside a patient. By comparing the phase values of signals received at two or more energy sensors near the treatment location, where the energy sensors may be positioned in a known positions within one or more cavity and/or lumen, and knowing distances between the energy sensors, adjustments to the transmission phase values for all elements of the phased array may be computed automatically. For example, the projected energy beam focal spot is positioned near the energy sensors with an accuracy of 100 micrometers. The energy sensors may be introduced near the treatment region using a catheter, endoscope, and the like.

According to some embodiments of the present invention, there are provided methods and devices for utilizing energy sensors in proximity to a treatment location to track the treatment location during an ablation procedure automatically. As in the multiple stage example described previously, the first and second stages may be repeated prior to each treatment energy beam projection, and for each element of the phased array transducer the transmission phase values of these repeated stages combined with the transmission phase values for each element determined during the initial focusing of the energy beam. This combination may allow adjusting the aim of the energy beam focal spot to track the target treatment location motion.

Optionally, the methods describe herein are implemented on any computerized device. For example, the methods are implemented on a computer, a digital integrated circuit, on an embedded microcontroller, on a client computer, on a server computer, and the like.

Optionally, ablation treatments are performed in conjunction with an imaging modality. For example, an ablation treatment is performed together with fluoroscopy in a catheterization laboratory, interventional radiology, projection radiology, x-ray imaging, digital radiography, magnetic resonance imaging, computed tomography, ultrasound imaging, and the like.

Optionally, one or more energy sensors are used in attached to a catheter, catheter guidewire, endoscope, wireless capsule endoscope, hypodermic needle, biopsy needle, and the like. For example, the energy sensors are positioned on a catheter guide wire and expanded from the catheter sheath at the target location so the energy sensors are positioned flush with an internal cavity and/or lumen of the patient.

Optionally, one or more energy sensors are able to detect the specific form of energy from a phased array energy projector. For example, the energy sensors are pressure sensors to detect energy for a HIFU transducer. For example, the energy sensors are electromagnetic radiofrequency sensors to detect energy for an electromagnetic radiofrequency energy transmitter. For example, the energy sensors are gamma radiation sensors to detect energy for a gamma radiation energy source.

Optionally, the ablation treatments described herein are performed for on any clinical indication on any part of a patient's anatomy. For example, an ablation treatment is performed for renal denervation on one or more renal arteries, heart arrhythmia ablation, prostate tumor ablation, spinal cord tumor ablation, brain tumor ablation, celiac plexus celiac ganglion, mesenteric plexus, carotid body, and the like.

Optionally, ablation treatments are performed by projecting a focused energy beam from a phased array energy projector to one or more discrete calculated points to perform the ablation treatment in one or more point-by-point patterns.

Optionally, ablation treatments are performed by projecting a focused energy beam from a phased array energy projector to one or more locations by sweeping the energy beam along a calculated path to perform the ablation treatment in one or more sweep patterns.

Optionally, ablation treatments are performed by projecting an energy beam from a phased array energy projector to one or more locations by intentionally defocusing the energy beam and increasing the beam energy and/or treatment time. This intentional defocusing allows shaping the energy projection focal spot to match the ablation treatment needed. This defocused energy projection may be used together with the point-by-point and/or sweep ablation patterns to perform the ablation treatment.

Optionally, ablation treatments are performed by projecting a focused energy beam from a phased array energy projector in a focal point shape best suited to perform the treatment. For example, in renal denervation the energy beam is focused in a crescent shape surrounding the artery and the energy projection is swept along the length of the artery multiple times at multiple locations. For example, in renal denervation the energy beam is focused in an arc shape surrounding the artery and the energy projection is aimed at points along the length of the artery one or more times at one or more locations.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

According to some embodiments of the present invention, there are provided methods to determine the focusing, aiming and tracking of energy from a phased array energy projector and/or a high intensity focused ultrasound (HIFU) transducer. The projected energy beam may be used to perform an ablation treatment. For example, the methods allow performing a renal denervation procedure to treat hypertension with HIFU energy.

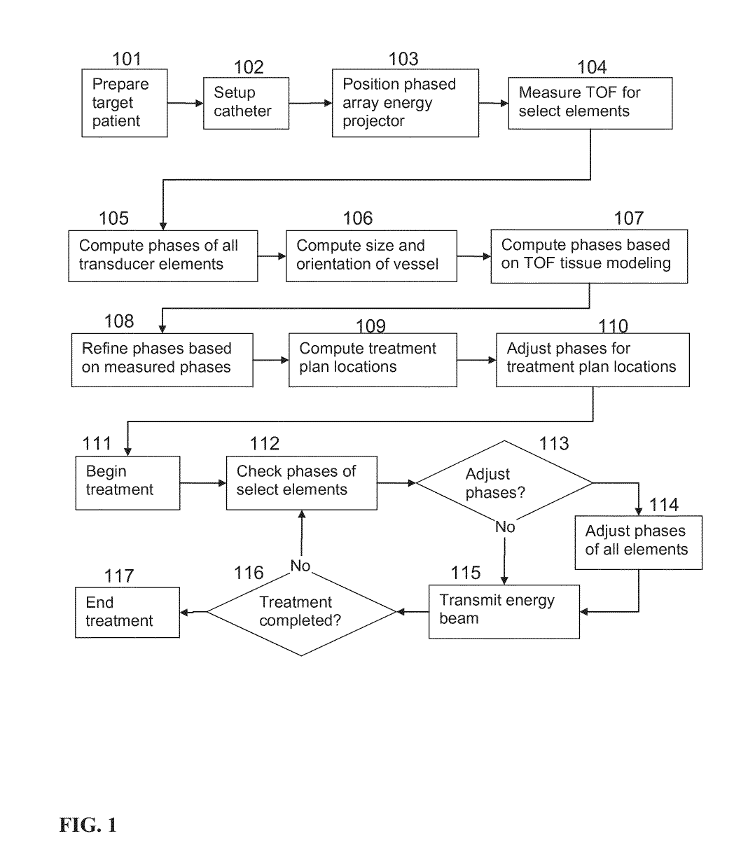

Reference is now made to FIG. 1, which is a flowchart of a method for renal denervation, according to some embodiments of the invention. The implementation of some of the methods for renal denervation may be initiated with the patient preparation in the catheter laboratory as at 101 and setup of the intravenous catheter as at 102. Subsequent to this the HIFU transducer may be positioned in contact with the patient as at 103. To allow focusing of the energy projection at the target treatment location, a three stage approach may be used according to some embodiments of the invention. The first stage may project energy from one or more selected phased array elements and may measure TOF values as at 104 of the signal received at the energy sensors. The TOF values are used to compute phases for all phased array elements of the energy projector using tissue modeling techniques as at 107. Stages two and three both comprise projecting energy from one or more phased array elements and collecting phase values as at 105 from the signal received at the one or more energy sensors. The difference between stage two and three being that in stage two only representative phased array elements project energy to produce phase values and these representative phase values are used for determining phase of all elements of the phased array energy projector, for example by means of interpolation, extrapolation, or any other calculation. In stage three all phased array elements project energy to produce phase values to be used directly be each phased array element, and stored for adjusting their determined phase according to the previous stage. Optionally, the system uses laboratory calibration and/or mathematical calculations instead of stage three and only two stages are performed. Optionally, stage one is replaced by determining a rough estimation, whether by measurement of an imaging system, automatically, or manually, by human input, by a calculated guess, or by a constant, thereby eliminating stage one. These three stages each produce a phase value for each phased array element of the energy projector as at 108, which are combined to produce the phase value used for focusing the HIFU energy beam. Performing the phase calculations in three stages, where the first and/or second stages may be performed in a relatively short time allows motion tracking during treatment.

By collecting one or more signals from one or more energy sensors from the same projected energy of one or more phased array projector elements allows computing a phase map for the target treatment location which may be used to aim the focused energy beam. The size and orientation of the vessel may be computed as at 106, and the treatment ablation locations planned as at 109. The aiming determined in 108 s then adjusted to target the treatment locations.

Once treatment has begun as at 111, a continuous process of first checking the phases of selected phased array elements to determine tissue motion effect on the phases of the transmitter elements, as at 112 may be performed prior to transmission of the HIFU energy beam as at 115. When the phases measured at 112 have changed since the last time frame as at 113, adjustments are made to the phased array element transmission parameters to account for the target tissue motion as at 114 before transmitting the HIFU energy as at 115. When the treatment has been completed as at 116 then the procedure may be terminated as at 117.

Aiming a projected energy beam to a target treatment location using linear geometry assumes homogenous acoustic velocity in the tissue medium. Such assumed homogenous acoustic velocity may create distortions causing the focus to be displaced from the intended target location, and blurring of the treatment boundaries. Such blurring of the treatment boundaries may be seen as a defocusing of the beam, may cause the focus area to have lower energy concentration per volume, which may be less effective in an ablation treatment.

In phased array transducers, whether ultrasonic or electromagnetic, the transducer may contain a large number of phased array elements, and focus a projected beam of energy by adjusting the amplitude and phase of each phased array element. The phase of each phased array element may be determined from the phase map at the target tissue location. For example, by inserting an energy sensor into the renal artery to be denervated, the phase map may be measured directly in close vicinity to the target region. Using this acoustic map, when the energy projector uses ultrasonic energy, the phases of each phased array element may be adjusted to focus the energy beam at the target treatment location, and the target treatment volume accuracy increased significantly. This may result in reduced target volume per energy projection and sharper ablation volume boundaries when compared with methods not utilizing energy sensors at the target treatment location. The energy sensors at the target treatment location also may allow accurate mapping of the size and direction of the artery, lumen, and/or cavity so the ablation process may avoid damage to healthy tissue. Furthermore, the energy sensors at the target treatment location may track phase map changes due to physiological and patient motion in real time during the ablation procedure and may allow locking the ablation volume location relative to the energy sensors by adjusting the phases of the transmission for each element. This energy beam aim tracking may result in decreased treatment time, increased outcome effect, decreased resurgery and decreased adverse effects.

In a similar manner, placing one or more acoustic sensors and/or energy sensors on the vicinity of the target tissue, may assure these benefits for other type of medical ablation treatments. For example, inserting acoustic sensors to the area of treatment by positioning these sensors on a distal tip of an intravascular catheter during treatment may allow phase map calculation. The position of the sensors may be determined by the positioning of the catheter tip during treatment. For example, inserting a catheter to a position in the vicinity of a treatment location is performed using a catheter laboratory for fluoroscopic imaging of the relative positions of the catheter, patient treatment location and phased array energy projector. The fluoroscopy may ensure that the catheter tip and/or energy sensors are sufficiently close to the treatment location to enable accurate phase mapping and that the ideal focus location of the energy projector may be centered at the target treatment location. For example, the fluoroscopy images are used to position one or more energy sensors in a renal artery using a catheter. The energy sensors may allow accurate phase mapping of an ellipsoid region that has a 3 centimeter major axis and 1.5 centimeter minor axis, and the energy sensors are positioned within the renal artery 5 millimeters from the target treatment location. For example, the fluoroscopy images are used to position an extracorporeal phased array HIFU transducer touching the patient's skin as close as possible to the target area as viewed on the fluoroscope. The energy projector may be positioned between the ribs and pelvic bone of the patient at a 45 degree angle from the medial-lateral axis so that the energy beam may be focused at the treatment location surrounding the renal artery.

Additional specific applications of the invention may include other treatment areas of the anatomy that are difficult in aiming and/or tracking a projected energy beam, such as within the rib cage, pelvis, spinal cord or skull. For example, liver tumor ablation, heart arrhythmia ablation, prostate tumor ablation, spinal cord tumor ablation, and brain tumor ablation. Optionally, other treatment areas include targets close to blood vessels and/or cavities of the anatomy.

The system comprises a catheter guided extracorporeal high intensity focused ultrasound (HIFU) system for the treatment of hypertension by renal nerve ablation. The purpose of the device may be to safely and effectively ablate the afferent and/or efferent nerves around a treated renal artery using ultrasound energy. The ultrasound energy may be transmitted from the system's transducer. The transducer may be positioned outside the patient, touching the skin at a posterior-lateral position, at an approximate 45 degrees angle, such that the path of the energy does not cross the spine. The endovascular catheter may be a passive device, which enables accurate tracking of the treatment area allowing precise positioning of the ultrasound focus volume at a path around the artery, and not inside it.

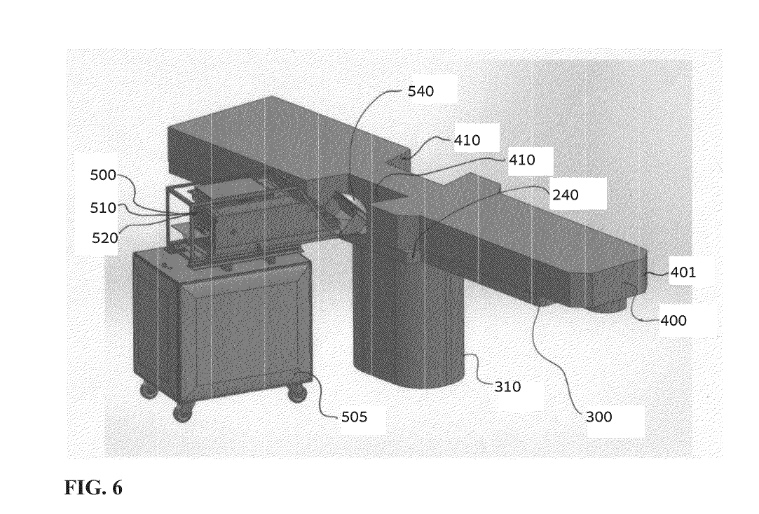

Reference is now made to FIG. 6, which is a schematic illustration of some systems for renal denervation, according to some embodiments of the invention. The system may consist of four main components: catheter, transducer as at 540, control unit as at 505, and mattress as at 400. The HIFU transducer may be connected to power transmission electronics as at 500, which are controlled by an electronics controller as at 510 and interface with the control unit as at 520. The mattress contains two recesses as at 410, one on either side of the patient, which allow the transducer to be positioned against the patient during treatment. The mattress may have rounded corners as at 401, and rests on the surgical table as at 300 which may be supported by a pedestal as at 310.

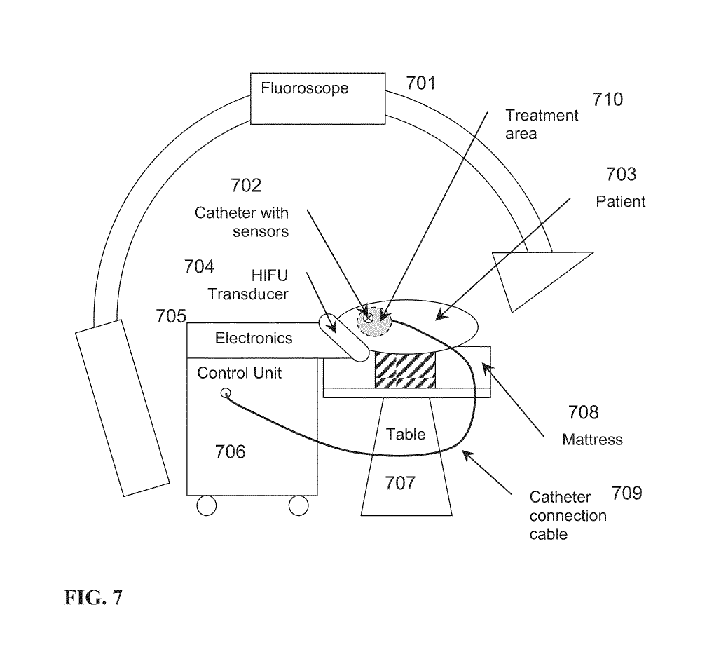

Reference is now made to FIG. 7, which is a schematic illustration of some systems for renal denervation in a catheterization laboratory, according to some embodiments of the invention. The illustration shows the position of the fluoroscope as at 701 used to align the catheter 702 and transducer 704 with the target treatment location 710 inside the patient 703. The catheter may be connected to the control unit 706 using a connecting cable as at 709. The mattress has recesses 708 to allow the HIFU transducer to be positioned on the posterior and lateral side of the patient at an approximate 45 degree angle with the coronal plane, in other words rotated 45 degrees about the inferior superior axis as illustrated. The HIFU transducer may be powered by electronics 705 which are in turn connected to the control unit 706.

The system may consist of four main components: catheter, transducer, control unit, and mattress.

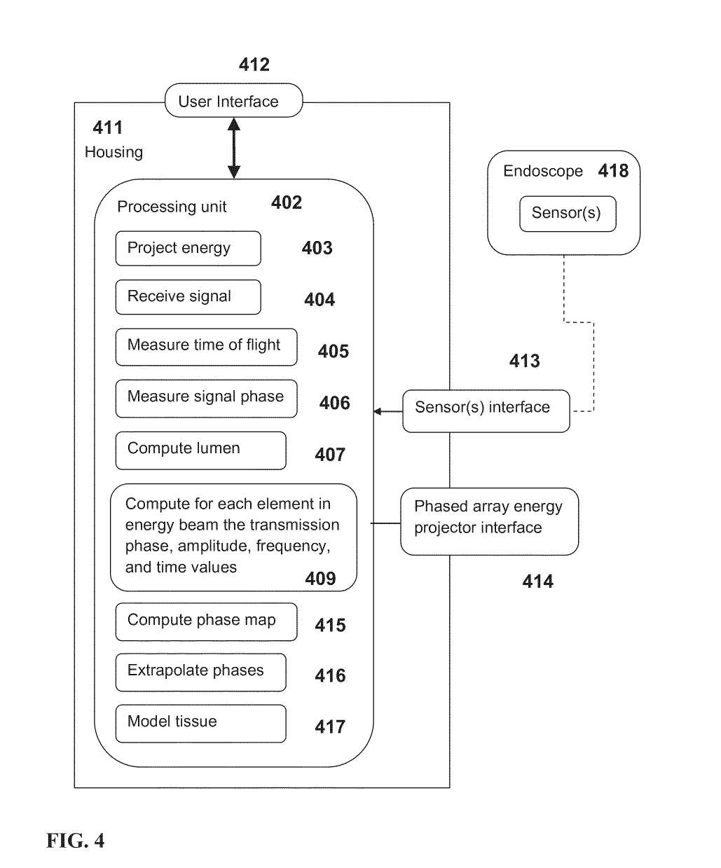

Reference is now made to FIG. 4, which is a schematic illustration of a computerized device for renal denervation, according to some embodiments of the invention. The device may be contained in a housing as at 411, contains one or more processing units 402, one or more user interfaces 412, an interface for one or more sensors 413, and an interface for the phased array energy projector 414. The one or more processing units are configured for performing the action of projecting energy 403 using the phased array energy projector interface 414. The one or more processing units are configured for performing the action of receiving one or more signals 404 using the energy sensor interface 413. The one or more processing units are configured for performing the action of measuring a TOF value 405 from one or more sensors signals. The one or more processing units are configured for performing the action of measuring a phase value 406 from one or more sensors signals. The one or more processing units are configured for performing the computing a lumen size orientation and location 407 from one or more phase and/or TOF values. The one or more processing units are configured for performing the action of computing beam focus parameters 409 from one or more TOF and/or phase values. The one or more processing units are configured for performing the action of computing a beam aim parameter 409 from one or more TOF and/or phase values. The one or more processing units are configured for performing the action of computing a phase map 415 from one or more phase values. The one or more processing units are configured for performing the action of extrapolating phase values 416 from one or more phase values 409. The one or more processing units are configured for performing the action of modeling tissue 417 from one or more TOF values.

The ablation system may be located in a catheter lab, and comprises a HIFU-enabled patient positioning/treatment table, HIFU transducer, and catheter with acoustic sensors. When a patient is ready for ablation, and positioned on the table, the catheter may be inserted into the treatment area. Correct location of the catheter and positioning of the patient may be performed using fluoroscopy which displays the location of the catheter, the treatment area and the table position using a ruler visible to the operator from the lateral aspects of the table. Subsequent to the correct catheter positioning as determined by the fluoroscopy, the phased array transducer may be placed against the patient and the system performs an element by element transmission of low powered ultrasound pulse from all phased array elements of the HIFU array, and detects the pulse signal using the catheter based sensors. Based on the phase of each element's pulse received at the sensors, a map of the phased array element phases may be produced that enables accurate aiming of the HIFU energy. By performing this process on a subset of representative phased array elements during the treatment stage, and adjusting the phases of transmission of all phased array elements accordingly, the relative motion and/or displacement of the tissue volume may be tracked to keep the focal point of the energy at the target treatment location relative to the one or more sensors.

The initial aiming of the phased array energy transmission may be performed with three major steps in a three stage approach. The first stage may be to estimate the expected phases for each element of the phased array energy projector. This step may be by an initial estimation of an average speed of sound and an estimated location. An estimated location may be reached by use of a human estimation, imaging device estimation, constant estimation, TOF-based tissue modeling estimation, and/or other techniques for conversion of time values to phases of each phased array element for focusing the energy beam. The second stage may be used to measure a set of representative phased array elements, or a set of groups of such elements. The group may be any collection of phased array projector elements. For example, the group is a collection of nearby phased array elements. The third stage may be used to measure each individual phased array element or group of elements, based on a measured relative phase value. For example, position calculations are not computed from the catheter sensors, such as conversion of like TOF values to distance values, and only phases values are used in estimating the phased array element phases that focus the projected energy beam.

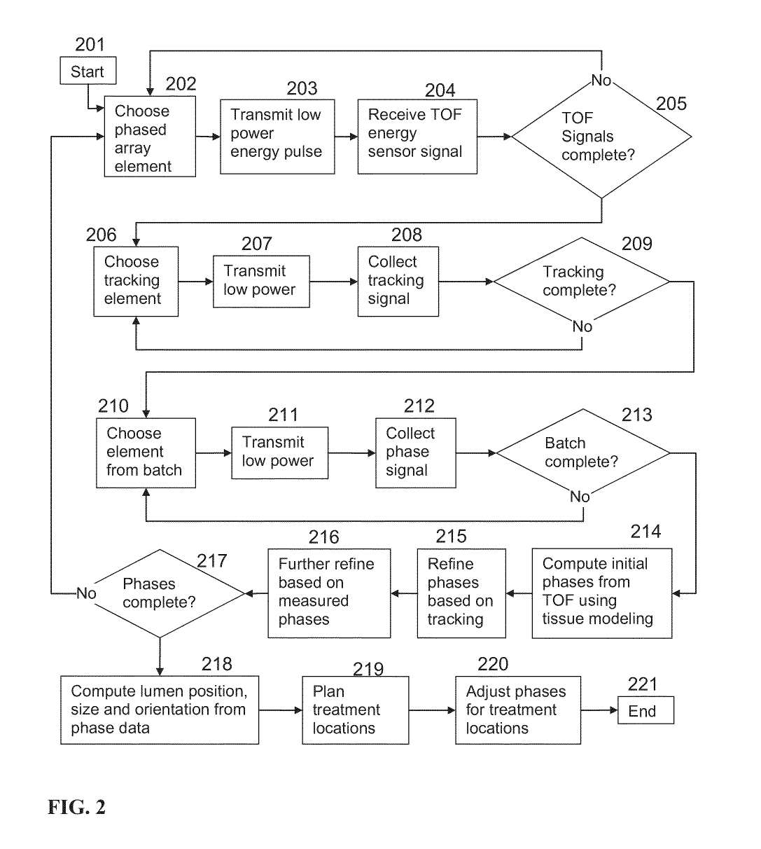

Reference is now made to FIG. 2, which is a flowchart of a method for focusing and aiming a phased array energy projector, according to some embodiments of the invention. Subsequent to starting 201 the method for initial energy transmission phase determination may proceed in three stages. During the first stage, one phased array element of the TOF element group may be chosen 202, low power energy may be transmitted from that phased array element 203, and a signal collected for TOF value calculation 204. This may be repeated till all phased array elements of the TOF group are been processed 205 and the first stage may be complete. The second stage chooses a tracking phased array element 206 from the tracking group, transmits low power energy from that phased array element 207, and collects a sensor signal for phase value calculation 208 until all phased array elements of the tracking group are have been processed 209. Optionally, these tracking group phased array elements are changed to new phased array elements during the HIFU energy beam projection. For example, when a sensor signal is not detected from an energy pulse transmitted by a phased array element, this phased array element, possibly blinded, is replaced with a non-blinded phased array element. During the third stage transmission phases are determined for all phased array elements of the transducer in batches. Each batch may be preceded by a repeat of stages one and two to correct for target motion between batch collections. During the collection of one batch, a phased array element may be chosen from the batch 210, low power energy may be projected from that phased array element 211, and a sensor signal may be received to calculate a phase value for that phased array element 212 until the batch may be complete 213. The batch and stages one and two comprise a time frame. For each time frame, the initial phases are computed from the TOF values using tissue modeling 214, phases are refined based on measured phases of the tracking group of phased array elements 215, and the phases of the batch phased array elements are used to further refine the transmission phases of the transducer 216. This allows computing the phases for all batches and correcting for patient motion during the initial focusing. Once the transmission phases for all batches have been computed, the lumen size and orientation may be computed 218 from the phase and/or TOF values from multiple sensor signals. The treatment ablation plan determined 29 and the phases adjust for the treatment ablation patterns 220. This completes the focusing and aiming of the projected energy beam 221.

Together these three steps allow better positioning of the ablation focus, smaller size of the ablation focus, and shaper boundaries of the ablation focus. Since the system localizes the HIFU focus relative to sensors, there may be no need to know the absolute position of the sensors.

Detection of the TOF signal based on the received signal may need methods dedicated to the specifications of the energy projector and energy sensors. For example, the time response of the projector and sensors may be taken into account. For example, the signal to noise of the energy sensors may be taken into account. Any given received signal may show background noise prior to arrival of a pulse, a ramp up of the pressure modulation amplitude envelope due to the transducer and energy sensor time responses, the modulations reach peak amplitude, and subsequently decay back to zero. The difficulty in detecting the time of flight measurement may be that the received signal contains noise, a modulating sine wave, and the transducer and/or sensor ramp up and ramp down delays. This makes the signal amplitude at time zero of the received signal smaller than the noise amplitude. To get around this issue, dedicated algorithms may be used that look at the windowed cross correlation function between the transmitted sine wave and the received signal, the auto correlation function of the background noise, and/or perform modeling of the transducer and hydrophone responses.

To allow better TOF modeling, only phased array elements that reach target with sufficient amplitude are chosen for TOF value measurement, both at the initial setup stage and during the motion tracking stage. During the initial setup, all phased array elements are tested to determine when they are blinded to the target location by sending pulses from each phased array element and measuring the peak amplitude of the signal reaching the catheter sensors. A small number of phased array elements are selected throughout the array to use for TOF measurements such that they have the highest received signal. They are chosen so as to be approximately evenly spaced around the array and they circumscribe as much area of the array as possible.

To convert time of flight into phases a cost function may be used, described by the equation:

$$CF = {\sum }_{i} \vert {T}_{i} C - \vert {\vec{x}}_{i} - \vec{r} \vert \vert$$

where i denotes the index of all phased array elements used for tissue modeling, Tᵢ denotes the measured time of flight of phased array element i, c denotes the approximated speed of sound in the tissue, \vec{x}ᵢ denotes the position of phased array element i in the array, and \vec{r} denotes the sensor position being sought that minimizes the cost function.

Once the sensor position \vec{r} has been determined, the phases of all transducer array elements may be computed using tissue modeling so that the focal spot may be centered on the sensor position. These estimated phases may be used as a starting point for the further phase corrections in the subsequent steps.

The method determines TOF values from enough phased array elements and different areas of the transducer to get initial phase estimates for focusing, while doing it in a short duration. For example, the element phases for beam focusing are calculated from TOF values in less than 10 milliseconds.

Using short time cycles for energy transmission during TOF measurements may be favorable for reducing noise from the echoes and thus increases the accuracy of the measurements.

In contrast to the technical issues in detecting the TOF signal zero time, there are fewer issues in detecting phases between the phased array elements. For example, the relative phase between the phased array elements of the transducer may be measured by modeling a sine wave to the large amplitude signal region, and an absolute value of the phase arriving at the transducer may not be determined. It may be sufficient to compute the relative phases between the transducer phased array elements to allow focusing of the phases so that they arrive aligned at the sensor.

By choosing a fixed time point during the envelope of peak pulse signal amplitude, the phase of the pulse may be detected during this time by correlation with a sine function and solving for the phase that best matches between the received signal and an ideal sine function. This relative phase correction may be stored for each phased array element, and applied to the tissue based modeling phases to better focus the HIFU energy.

During the second stage of the initial phase calibration, selected phased array elements are chosen from each quadrant of each group of phased array elements. Enough group binning may be done on the phased array elements so as to determine phases with sufficient accuracy. For example, when the transducer is constructed from phased array element groups manufactured on one substrate, choosing a phased array element from each quadrant of each array group would allow sufficient and uniform coverage of the complete array for accurate determination of the phase corrections. The time of the transmitted energy pulse from each phased array element may be small and thus the number of cycles used with each phased array element may be small. Thus the total duration of measurement may be small, may be done fast enough to be performed concurrently with the HIFU energy projection and thus enable target location tracking.

In the third stage of the initial phase calibration, the phase of each element of the array may be measured directly. This may be done in batches of large groups of phased array elements randomly selected from the array, and interleaved with the first and second steps before each group. Each of these sequences of large number of groups may be preceded by the first and second step, in what may be referred to as a time frame. In this manner, the energy sensor motion may be tracked during batches of individual element phase measurements so that motion may be monitored and corrected for.

Optionally, stage two tracking phased array elements are from a group of random phased array elements, distributed phased array elements, patterned phased array elements, random phased array elements chose from physical groups, random phased array elements from selected groups, and the like. Optionally, the phased array elements for each tracking cycle are all or in part from the same group as a previous cycle. Optionally, the phased array elements for each tracking cycle different from a previous cycle, all or in part. For example, using random phased array elements from the phased array energy projector elements that are not blinded to the target location avoid compounding errors. For example, part of the phased array elements used for tracking are known good elements and the other part selected from random groups of phased array elements.

The catheter tip may contain one or more energy sensors positioned along a helical spring designed to expand to be close to the inner wall of the vessel lumen once extended from the catheter. Thus the energy sensors may be situated along the lumen wall at known interval distances along the helix. By computing the phases of the signals received at each energy sensor from an ultrasound energy pulse, the physical parameters of the lumen that contains the sensors may be determined Measuring time of flight between transducer and sensors may not yield accurate position due to non-homogeneous speed of sound in tissues en-route to the target area, and therefore orientation calculation for determining the vessel direction may become erroneous. Measuring phase differences in small areas of relatively homogeneous tissues may enable the accurate computation of short distances, and orientation.

Due to the fact that the sensors are relatively close one to the other and that the medium between them may be relatively homogeneous, phase value calculations may be equivalent to distances in between sensors and may yield very accurate phase map results. For example, the energy sensors are a few millimeters distanced one from the other and the measurements are determined with accuracy of about 100 microns. Thus with these parameters known, the ablation treatment may be planned so as to effect the nerves surrounding the blood vessel with minimal damage to non-target tissue and organs. For example, the known distance between the two or more sensors and the measured phase differences give an accurate speed of sound measurement at the target treatment location, taking into account the orientation of the lumen. By using this measured tissue speed of sound velocity and computing a phase map of the surrounding tissue the phase adjustments needed for all projector phased array elements for reaching a target treatment location in the surrounding tissue may be accurately determined

Optionally, time of flight and/or phase values are used for modeling the positions of the energy sensors and finding the best fit cylinder passing through all of the positions. The TOF and/or phase measurements may be used to compute the position of the center of the helix relative to the transducer, the size of the lumen, and the direction of the lumen.

Optionally, phase calculations are done for a nearby estimated location, without calculating element phases with a speed of sound value. For example, calculating a TOF value does not calculate a position because of variations to speed of sound. Using TOF values with estimated, approximated and/or measured average speed of sound in the tissue to calculate a target position relative to the energy projector, and then using the calculated position to estimate the phases of the phased array elements needed to produce a beam focus at that position compounds the errors in the velocity of the beam in the tissue and the resulting focal point may be large and blurred.

Optionally, treatment path is determined relative to a sensor, or sensors near a target tissue, but directions of that path are determined relative to the transducer. For example, path may be determined to go from greater than or equal to 2 millimeters from a sensor to 12 mm and greater, thereby not needing to calculate the direction of tissue.

During energy projection for the ablation treatment, time frames are used to cycle between energy projection and tissue cool-off time periods. Prior to each energy projection, the target treatment location may be tracked with a series of measurements from the one or more projector elements such that the time for the target tracking may be short relative to the time frame and may be performed at sufficient temporal resolution to track the target accurately. The target tracking may perform the first two stages of energy beam focusing and may use the relative phases of each phased array element determined previously.

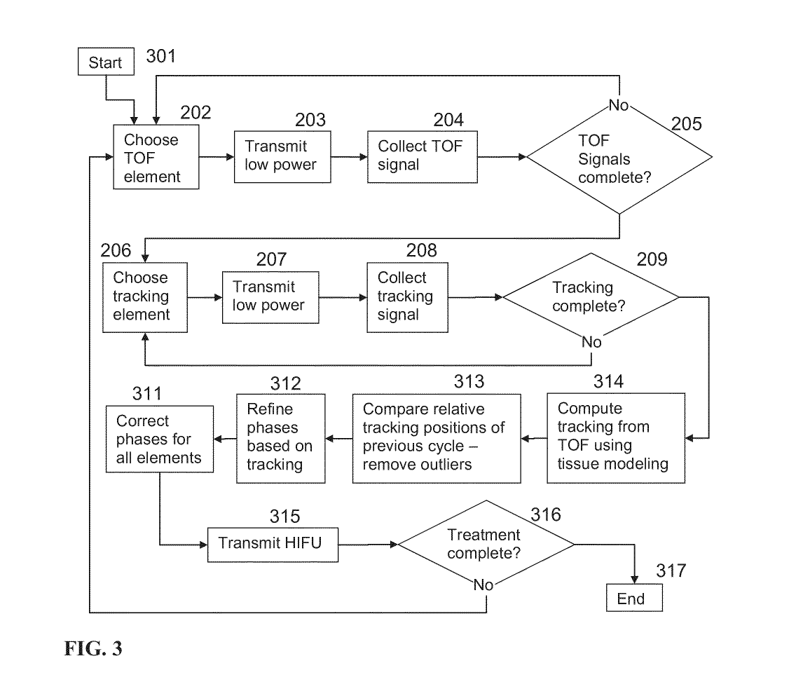

Reference is now made to FIG. 3, which is a flowchart of a method for tracking a treatment location to aim a phased array energy projector, according to some embodiments of the invention. At the start of each time frame for HIFU energy transmission 301, the same first two stages as performed in the initial focus and aiming are repeated as at steps 202 thru 209.

For TOF measurements a small representative group of phased array elements may be used. These may be chosen from the group of elements throughout the array with highest sensor signal amplitude, such that the phased array elements are distanced from each other and around the edges of the transducer array. The new tracking TOF values are used to compute tracking based on tissue modeling 314, data value outliers may be removed based on TOF and phase values 313, the phases refined based on the TOF computed phases, and interpolated direct phase measurements, the transmission phase for all phased array elements computed 311, and the HIFU energy transmitted 315. This process may be repeated as at 316 till the treatment may be complete and the procedure terminated 317.

Optionally, for every time frame a different set of phased array elements for TOF measurements is used and the calculations are done for all other phased array elements of an energy projector.

Optionally, the maximum signal amplitude is monitored during the tracking stage. When there is a consistent decrease in the signal due to motion-induced disruption of the beam path and the signal attenuation at the sensors, a new phased array element may be chosen from the energy projector for the representative TOF and/or phase value phased array elements. Optionally, any set of energy projector elements may be used for TOF and phase value measurements. For example, the representative set of phased array elements is chosen from a fixed set of phased array elements, cyclic choose, one more is randomly choose, and the like. Optionally, the sets of phased array elements used for TOF values and phase values are different. For example, the TOF values are calculated from a few representative elements around the energy projector periphery, and the phase value set of elements is distributed throughout the energy projector array.

Optionally, during the HIFU energy transmission and tracking stage only the TOF modeling and/or phases of representative phased array elements are monitored for target location motion. These measurements may be used to determine when a change in transmission phase value may be needed for reaching the target tissue due to motion.

Since the transducer phased array elements are rigidly fixed in relation to each other, this may be sufficient for motion tracking when combined with the relative phases measured from each individual phased array element.

Optionally, there is no expected motion of the target tissue, and measurements are conducted only at the initial stage for accurately determining the phases required for the treatment path.

Optionally, in addition to tracking the relative location of the sensors, the method also uses previous time frame tracking signal parameters, such as amplitude and phases of the sensor signals, to compare with the current signal, and thus deduce when there has been a movement of the target location. When there is a big discrepancy between these parameters, the system may selectively redo all or part of the measurements to confirm the motion. Optionally, the system may continue tracking the motion until the parameters indicate a relatively stable target location. For example, when there has been a jerking motion according the parameters, then the system continues monitoring the motion until the patient has stabilized before continuing the ablation procedure to avoid erroneous position of the treatment volume.

Together, the TOF and phase methods may give good estimation of the motion in a patient, whether it be fine motion, for example due to breathing, or coarse motion, for example due to a patient muscle spasm, patient tremor, patient shudder, and the like.

Differences in phase from a previous cycle may be used to detect motion of the sensor without detecting the sensor absolute position. When large motion is detected the system may stop the HIFU energy projection and wait for the patient motion to stabilize. Optionally, only the phase method is measured, and used for correcting HIFU phases, without the use of TOF or any other location. Optionally the previous set of phases is used to determine the difference between the previous time frame to the current time frame, and algorithms of interpolation, extrapolation and/or the like are used to determine the effect on each element transmission during HIFU.

One of the advantages of the present invention may be the capacity to overcome speed of sound differences in individual tissues and/or patients for a coherent focus at a very accurate target location relative to the energy sensor/s position. An additional advantage may be the capacity to update the projected energy beam aiming according to one or more energy sensor signal calculated values in high frequency and therefore move the path of treatment relative to the tissue sensor, which may be anchored to the target treatment location. For example, the aiming is updated at a rate of 3 Hz, or 1 Hz, or 10 Hz, or 100 Hz, and the like.

The methods described here are based on catheter-based acoustic sensors that assist in performing the described functions. Optionally, the catheter contains other sensors and/or for performing these tasks. For example, the catheter contains one or more temperature sensors, one or more navigational positioning sensors, and/or one or more transmission devices to assist in performing these functions.

Optionally the sensors are not positioned on a catheter, but on another device—such as an endoscope.

Optionally, different phased array energy projectors may create ultrasound focus, such as single element transducers, annular array transducers, acoustic lens transducers, phased array transducers, etc.

Optionally, the sensors are transducers and/or transmitters. Optionally, there are multiple different elements such as transducers, sensors and/or transmitters.

As mentioned previously, additional specific applications of the invention might include other treatment areas of the anatomy where there might be difficulty in aiming and/or tracking HIFU energy, such as within the rib cage, pelvis, spinal cord or skull. Examples might include liver tumor ablation, heart arrhythmia ablation, prostate tumor ablation, spinal cord tumor ablation, and brain tumor ablation. Other neural ablations of the sympathetic system may include treatments such as celiac plexus celiac ganglion, mesenteric plexus, carotid body, and the like. In general, any anatomical location where a catheter or a needle, or any other device may be placed in close proximity to the tumor may benefit from the advantages of these methods. For example, the energy sensors are inside an endoscope capsule implanted in the brain near a brain tumor.

Optionally, primarily denervation is performed of other neural structures, and/or other organs.

In general, the advantages over previous methods are in the utilization of the catheter based sensors for better performing the ablation treatment. It is well know that the speed of sound in tissue depends on the type of tissue. These variations in speed result in aberrations of the ultrasonic wavefront, such that the resulting energy arrives at the target location at different phases modulated by the speed of sound of the multiple tissues that the sound wave travels through. Since the transducer contains a large number of small phased array elements, these phases of each phased array element may be corrected when the acoustic phase map in the target tissue is known. By positioning a catheter with acoustic sensors into the renal artery to be denervated, the acoustic map may be measured directly in close vicinity to the treatment target region, the phases of each phased array element adjusted accordingly, and the target treatment focus accuracy increased significantly. For example, calculations of phase maps from energy sensors at the target region decrease target focal spot size and increase sharpness of target focal spot boundaries. Due to the accuracy and the ability to correct for tissue motion of the methods described herein, the ability to conduct stripe ablations rather than point ablations may be dramatically increased enabling better patient safety and better treatment efficacy. For example, using a sweep ablation pattern produces homogenous thermal treatment rather than hot and/or cold spots. For example, using a sweep ablation pattern eliminates non effective treatment areas between ablation points. The catheter sensors may allow mapping of the size and direction of the artery so that the ablation process may avoid damage to the artery. Furthermore, using the sensors during the ablation procedure in real time to track physiological and patient motion allows locking the ablation volume relative to the artery to achieve an accurate, robust and consistent pattern of ablation around the artery. These advantages may result in decreased treatment time, increased outcome effect, decreased resurgery and decreased adverse effects.

The specific advantages of the methods for detecting the optimal focusing of energy and tracking the treatment location during ablation may be further exemplified. As most HIFU ablation treatment methods rely on administering HIFU energy for ablation from the catheter or completely non-invasively using an external HIFU transducer and non-invasive imaging methods, they are in concept and practice more limited in the effectiveness of the treatment. Catheter based HIFU energy delivery may be limited in the amount of energy transmitted as the size of the catheter may be limited by the blood vessel it is contained in. When the HIFU transducer is at the tip of the catheter closest to the tissue, then the size of the transducer may be limited as well as the heat produced from the piezoelectric element. When the HIFU transducer is at the other end of the catheter, outside the patient, then the acoustic transmission guide may be the limiting factor in the power delivery. In both cases there may be limited power for treatment, longer treatment times, and more side effects due to the large size catheter. Additionally, the ability to focus the treatment position from the edge of the catheter may be limited by the catheter size, and therefore the farther from the vessel, the lower the energy, therefore when longer distance may be needed, excess of energy may be disposed at the artery wall.

In non-invasive HIFU energy administration, the inaccurate positioning and large size of the focal spot of from the HIFU transducer may cause several disadvantages. For one, with a larger focal spot more tissue may be ablated causing more side effects to the surrounding tissue and organs. With less positional accuracy the margins of error need to be increased and the ablation treatment planned further away from the renal artery, thus needing more energy, more side effects, longer treatment times, and less optimal ablation treatment. Larger focal spots also need longer cool off periods as more energy has been deposited into the treatment area thus producing more heat. Additionally, the larger focal spot may also have less well defined edges, so there may be a partial treatment zone around each of the treatment locations. The most dramatic of all may be the fact that different tissues have different speeds of sound, which due to Snell's law, may distort the location of the beam focus, and displace it. At large distances from the transducer, these may reach a few centimeters of dislocation.

The sensors located at the treatment site also allow accurate pressure maps to be computed for the treatment area.

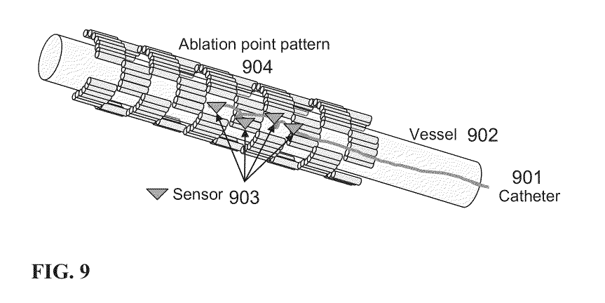

Reference is now made to FIG. 9, which is a schematic illustration of ablation point patterns for renal denervation, according to some embodiments of the invention. In this illustration the catheter guidewire 901 may be shown inside the vessel 902 to position the energy sensors 903 around the periphery of the vessel lumen. This may allow performing the ablation treatment in a point by point pattern around the blood vessel 904 so that the vessel may be denervated.

One aspect of the present invention may relate to the shape of the ablations conducted in renal denervation, or any other denervation or deactivation of neural structures. For example, the accurate focusing and aiming of the projected energy beam enables performing point by point ablations in a pattern so as to denervate the renal artery. Alternatively, the accurate focusing and aiming allows sweeping the focal point of the energy beam along predetermined paths surround the renal artery to perform sweep patterns of ablations.

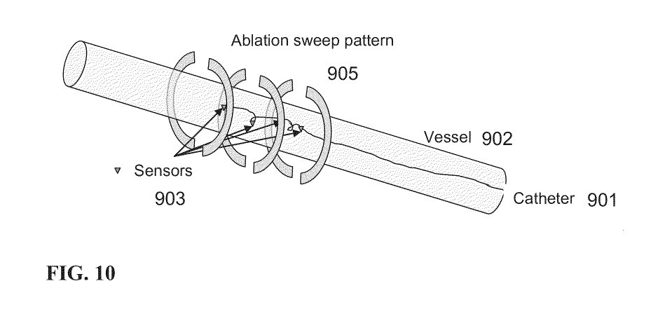

Reference is now made to FIG. 10, which is a schematic illustration of ablation sweep patterns for renal denervation, according to some embodiments of the invention. In this illustration the catheter guidewire 901 may be shown inside the vessel 902 to position the energy sensors 903 around the periphery of the vessel lumen. This may allow performing the ablation treatment in a sweep pattern around the blood vessel 905 so that the vessel may be denervated.

Using these high temporal resolution update capabilities may enable disposing energy at a target with a moving beam focus, such stripe pattern lesions may be created instead of point pattern lesions for ablation treatment. Such an approach may bring better patient safety results by not overheating the tissue at each point, and additionally may yield better efficacy for avoiding untreated target locations which may be created in point ablations without motion tracking.

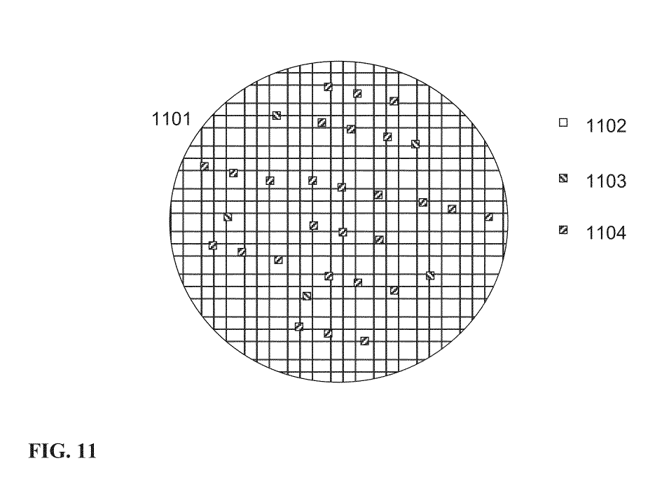

Reference is now made to FIG. 11, which is a schematic illustration of a phased array energy projector, according to some embodiments of the invention. The transducer 1101 may be a phased array HIFU transducer which may include thousands of transmitting phased array elements 1102 and may allow very accurate beam forming and precise electronic steering of the beam. Some elements of the phased array energy projector may be used to project a low power pulse for TOF measurements 1103 and some phased array elements may be used to project a low power pulse for phase value measurements 1104. The projected power pulses may be projected from a number of phased array elements in a series, each phased array element projecting a pulse after the pulse from the previous phased array element has been received at the energy sensor near the target treatment location. Optionally, a number of phased array elements project the energy pulses concurrently with different frequencies and/or waveform shapes between the pulses so that the sensors differentiate between the signals from the pulse projected from each phased array element. The beam focus location may be designed to follow automatically a predefined ablation path around the renal artery in stripes, and tracking any motion of the artery or patient without mechanical movement of the components. The transducer may be designed to couple in dorsolateral contact with the patient between the pelvis and ribs at an approximate 45 degrees angle in a axial cross section, and may be positioned after positioning the catheter in the artery.

The control unit may be a computerized component which controls automatically the catheter data and HIFU transmission. The unit may be connected to the catheter, receives energy sensor signals, and automatically analyze them. By analyzing the energy sensor signals, the computerized software algorithms may determine a beam path and focus location automatically. The control unit may be connected to the transducer, and automatically controls ultrasound transmission of the HIFU according to the above computations. The control unit may have a screen displaying beam path and ablation progress around the artery. The control unit may have a user interface to enable the operator to control and monitor the procedure.

The mattress may be compatible with standard catheterization beds, and may be designed to allow coupling of the transducer to the skin between the pelvis and the ribs. Reference is made to patent application number PCT/IB2012/054525, incorporated herein in its entirety by reference.

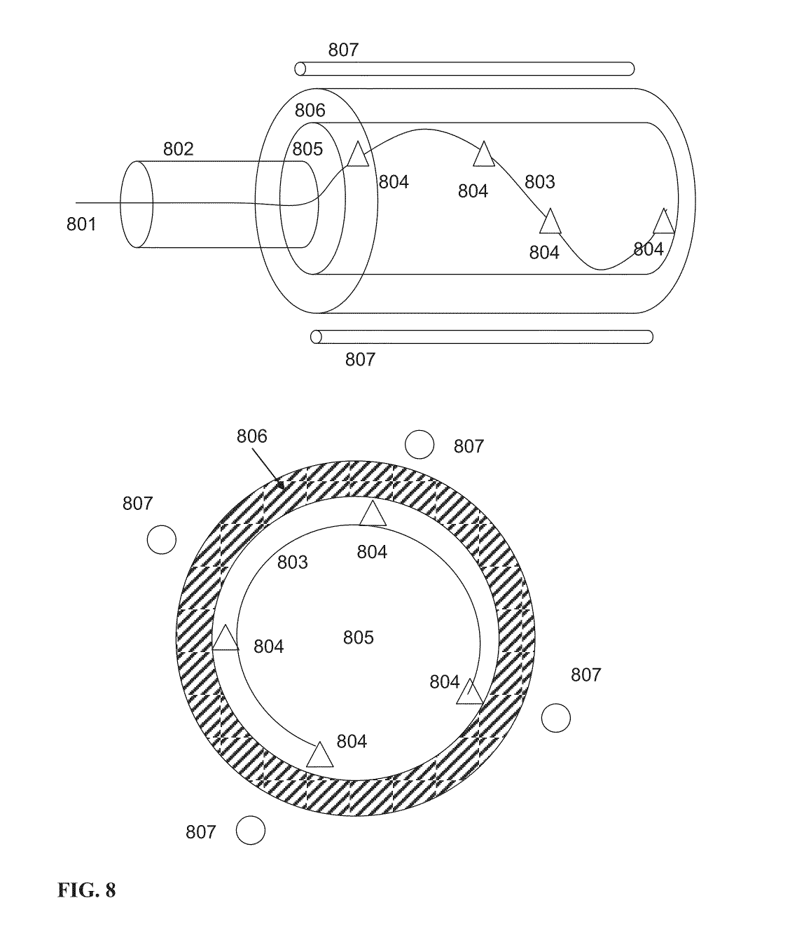

Reference is now made to FIG. 8, which is a schematic illustration of a blood vessel and catheter attached sensors for renal denervation, according to some embodiments of the invention. The illustration shows the catheter 802 extended from the catheter sheath 801. The helical portion of the catheter guidewire tip 803 contains one or more energy sensors 804. These expand to fill the blood vessel lumen 805 so that the energy sensors are positioned near the lumen boundary. His allows calculating the phase maps of the region around the sensor, including the target ablation nerve structures 807 that surround the blood vessel 806.

The catheter may be designed for single or multiple treatment use. The purpose of the catheter may be to enable precise targeting of the ablation location. The catheter may have an expanding conforming helical tip which may contain four pressure sensors. The catheter may be connected by electrical wires to the system's control unit. The catheter may be used with a 6 Fr guided catheter, and a 0.0014″ guidewire. The catheter may have two positions. The first position may be with the tip contracted inside the catheter's tube. At target position the tip may be expanded using the handle. The catheter may enable contrast media pass through. The catheter tip may have movable sensors that exit a sheath and may be located on a spiral helix. The helix may not contact with the vessel wall, which may be less traumatic for the vessel, but may expand to approximately fill the vessel lumen.

Optionally the sensors are not positioned on a catheter, but on another device—such as an endoscope. For example, an endoscope incorporates energy sensors for prostate tumor ablation treatment. Optionally, a wireless capsule endoscope incorporates energy sensors, and may be used for gastrointestinal tumor ablation treatment.

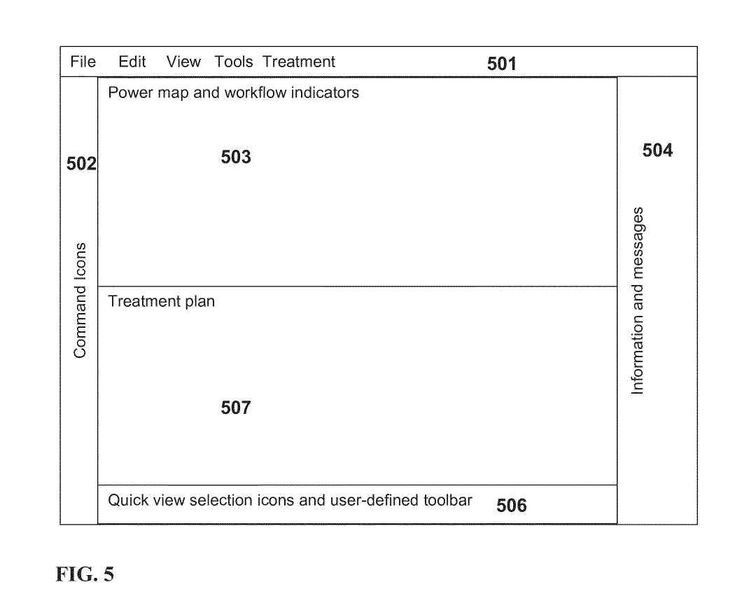

Reference is now made to FIG. 5, which is a schematic illustration of a graphical user interface for renal denervation, according to some embodiments of the invention. The user interface may contain a region of the screen for a menu list 501. The user interface may contain a region of the screen for command icons 502. The user interface may contain a region of the screen for a power map and workflow indicator 503. The user interface may contain a region of the screen for quick view selectors, shortcuts, and user defined toolbars 506. The user interface may contain a region of the screen for information and messages 504.

Optionally, the user interface uses messages and icons to indicated operator actions. For example, the system may automatically detect patient agitation by detecting excessive motion, send a message to the message area of the user interface, display a large alter icon on the workflow area of the user interface, and/or sound a indication alarm.

As used herein the term “about” refers to ±10%. The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for transmitting a phased array energy beam from an energy transmission group of elements of a phased array energy projector external to a body of a target patient, comprising:
transmitting at least one first energy pulse from at least one phased array element from a plurality of phased array elements of a phased array energy projector external to a target patient;
measuring at least one signal parameter of at least one first energy signal based on a reception of said at least one first energy pulse by at least one energy sensor;
calculating for each member of an energy transmission group of said plurality of phased array elements a first energy transmission parameter based on said at least one signal parameter;
transmitting at least one second energy pulse by at least one of said plurality of phased array elements;
measuring at least one signal parameter of at least one second energy signal based on a reception of said at least one second energy pulse by at least one energy sensor;
calculating for each member of said energy transmission group a second energy transmission parameter according to said at least one signal parameter;
calculating transmission instructions for transmitting a phased array energy beam toward an intrabody target area in a body of said target patient from said energy transmission group by combining said first and second energy transmission parameters for each member of said energy transmission group; and
controlling said phased array energy projector to transmit said phased array energy beam from said energy transmission group based on said transmission instructions.

2. The method of claim 1, wherein said first and second energy transmission parameters for each phased array element of said energy transmission group are any from the list of phased values, amplitude values, frequency values, time values, and the like.

3. The method of claim 1, wherein said transmission instructions for each phased array element of said energy transmission group are any from the list of phased values, amplitude values, frequency values, time values, and the like.

4. The method of claim 1, wherein said at least one energy sensor is placed in proximity to said intrabody treatment area of said target patient using any from the list of at least one catheter, at least one endoscopy, at least one wireless capsule endoscope, at least one hypodermic needle, at least one biopsy needle, at least one biopsy probe, and the like.

5. The method of claim 1, wherein said signal parameter of said at least one first energy signal is a measured phase value of said at least one first energy signal, where said plurality of phased array elements used to transmit said at least one first energy pulse is a subset of said energy transmission group, and said calculating is performed by extrapolation and interpolation of said phase values of said subset.

6. The method of claim 1, wherein said signal parameter of said at least one second energy signal is a measured phase value of said at least one second energy signal, and said at least one second energy signal is collected for all remaining elements of said energy transmission group.

7. The method of claim 1, wherein some of said signal parameters of said at least one first energy signal is a time of flight value of said at least one second energy signal, and said time of flight value is used tissue modeling to calculate a phase value.

8. The method of claim 1, wherein said at least one phased array energy projector is any type from the list of high intensity ultrasound phased array, radiofrequency electromagnetic phased array, gamma radiation phased array, proton therapy phased array, and the like, and said at least one energy sensor is configured to detect the projected energy of said phased array energy projector.

9. The method of claim 1, wherein said transmission instructions are calculated for transmitting said phased array energy beam toward an intrabody moving target area by combining previously determined transmission instructions with a repeated transmitting of said at least one first energy pulse, measuring said at least one signal parameter of said at least one first energy signal for a subset of elements of said energy transmission group.

10. A method for transmitting a phased array energy beam from a phased array energy projector external to a body of a target patient, comprising:
projecting at least one energy pulse from at least one element of a phased array energy projector located external to a body of a target patient;
receiving a plurality of sensor signals from each of said at least one energy pulses using a plurality of energy sensors located in a body of said target patient in proximity to an intrabody treatment area of said target patient;
measuring a phase value from each of said plurality of sensor signals;
calculating for each member of an aiming group of said at least one phased array elements at least one energy transmission parameter adjustment based on said phase values, at least one known distance between said energy sensors, and at least one relative position of at least one target treatment location;
calculating transmission instructions for transmitting a phased array energy beam based on previous transmission instructions and said at least one energy transmission parameter adjustment; and
controlling said phased array energy projector to transmit said phased array energy beam from said aiming group based on said transmission instructions.

11. The method of claim 10, wherein said at least one relative position is a plurality of relative positions defining at least one ablation point-by-point pattern.

12. The method of claim 10, wherein said at least one relative position is a plurality of continuous relative positions located along at least one ablation path defining at least one ablation sweep pattern.

13. (canceled)

14. The device of claim 23, wherein said at least one first signal parameter is a time of flight value.

15. The device of claim 23, wherein said at least one first signal parameter is a phase value.

16. (canceled)

17. The device of claim 23, wherein said central processor is adapted to: control said phased array energy projector for automatically aiming said phased array energy beam to at least one target treatment location in point-by-point pattern of ablation locations.

18. The device of claim 23, wherein said central processor is adapted to: control said phased array energy projector for automatically aiming said at least one phased array energy beam to at least one target treatment location in sweep pattern of ablation locations.

19. The device of claim 23, wherein said central processor is adapted to: control said phased array energy projector for automatically tracking at least one new target treatment location and automatically aiming said at least one phased array energy beam to said at least one new target treatment location.

20. The device of claim 23, further comprising at least one user interface displaying a three dimensional image of a ablation treatment plan in a coordinate system relative to a target treatment anatomy and said at least one energy sensor, enabling a user to instruct said device to perform modifications to said ablation treatment plan.

21-22. (canceled)

23. A device for transmitting a phased array energy beam from an energy transmission group of elements of a phased array energy projector external to a body of a target patient, comprising:
a projector interface adapted to send transmission instructions to a phased array energy projector external to a target patient;
a sensor interface adapted to receive a sensor signal from at least one energy sensor in proximity to an intrabody treatment area;
a central processor adapted to:
transmit at least one first energy pulse from at least one phased array element from a plurality of phased array elements of said phased array energy projector;
measure at least one first signal parameter of at least one first energy signal based on a reception of said at least one first energy pulse by said at least one energy sensor;
calculate for each member of an energy transmission group of said plurality of phased array elements a first energy transmission parameter based on said at least one first signal parameter;
transmit at least one second energy pulse by at least one of said plurality of phased array elements;
measure at least one second signal parameter of at least one second energy signal based on a reception of said at least one second energy pulse by said at least one energy sensor;
calculate for each member of said energy transmission group a second energy transmission parameter according to said at least one first signal parameter;
calculate transmission instructions for transmitting a phased array energy beam toward said intrabody treatment area in a body of said target patient from said energy transmission group by combining said first and second energy transmission parameters for each member of said energy transmission group; and
control said phased array energy projector to transmit said phased array energy beam from said energy transmission group based on said transmission instructions.
